(19) United States
(12) Patent Application Publication
(10) Pub. No.: US 2014/0141503 A1
(43) Pub. Date: May 22, 2014

(54) CELL CULTURE SUBSTRATE HAVING UNIFORM SURFACE COATING

(71) Applicant: Corning Incorporated, Corning, NY (US)

(72) Inventors: Yulong Hong, Painted Post, NY (US); Jennifer Lynn Hunt, Addison, NY (US); Meenal Pauddar Soni, Painted Post, NY (US); Hui Su, Elmira, NY (US); Jian Tan, Ithaca, NY (US)

(21) Appl. No.: 13/892,697

(22) Filed: May 13, 2013

Related U.S. Application Data
(60) Provisional application No. 61/728,591, filed on Nov. 20, 2012.

(51) Int. Cl. C12M 1/00

(52) U.S. Cl. CPC C12M 23/34
USPC 435/289.1; 427/256

(57) ABSTRACT

An article for culturing cells includes (i) a substrate having a surface; (ii) a plurality of pillars extending from the surface of the substrate; and (iii) a polymeric coating disposed on the surface of the substrate between the pillars, forming a plurality of mini-menisci on the surface of the substrate between the plurality of pillars. The mini-menisci may have diameters of from about 20 to about 250 micrometers. The plurality of pillars are spaced apart from one another in a manner to encourage cell growth on the mini-menisci rather than the on top of the pillars. Such articles may effectively address issues associated with the meniscus effect on a global scale.

This application claims the benefit of U.S. Provisional Application Ser. No. 61/728,591, entitled “Cell Culture Substrate Having Uniform Surface Coating,” filed Nov. 20, 2012.

### FIELD

The present disclosure relates to apparatuses for culturing cells; more particularly to cell culture apparatuses having a plurality of mini-menisci structures on which cells may be cultured.

### BACKGROUND

Cell or tissue culture on plastic surfaces, such as polystyrene, has been widely used to study cell behaviors, pathological mechanisms, drug potency, etc. It is generally understood that the more in vivo-like the cells behave in a culture environment, the more valuable information the study provides. However, cell culture on plastic surfaces fails to enable in vivo-like function or morphology of many cell types.

Recently, surface modification of standard plastic surfaces has led to improved in vivo-like morphology and function of cultured cells. Such modifications include modification of surface chemistry and mechanical properties (stiffness) using biological coatings (such as BD Bioscisence's Matrigel™, collagen, laminin, etc.) and synthetic coatings (such as Corning's Synthemax®, silicone hydrogels, etc.) which have achieved noticeable success for a variety of cell types. However coated culture plates can create mixed results during cell culture if there is an uneven coating.

One intrinsic physical phenomenon found when applying a polymeric coating on the bottom surface within a container, for example within a well of a culture plate, is the meniscus effect, which may result in either a concave meniscus or a convex meniscus. The meniscus is the curved upper surface of a coating caused by a difference in surface tension of the coating material and the material of the container or the object with which it is in contact. The curved coating surfaces caused by the meniscus effect can result in a number of issues including (i) heterogeneous cellular morphology, (ii) uneven cell distribution, and (iii) excessive amount of coating to attempt to avoid some of the issues resulting from the meniscus effect. Each of these three issues is briefly discussed below.

Heterogeneous cellular morphology may result due to differences in mechanical properties, such as stiffness, of the coating due to the meniscus effect. When the mechanical property (e.g., stiffness) of a coating material (such as soft hydrogel) is different from the underlying hard plastic (such as polystyrene) and only a limited quantity of coating or coating precursors is used, most coating materials accumulate near the edge of the bottom surface as a result of a concave meniscus effect. Even with an increasing quantity of coating material, the material tends to accumulate much more near or on the edge than at the center of the bottom surface. Therefore, the stiffness of the center is closer to the hard plastic substrate while the edge area assumes the stiffness of the coating material. The dynamic substrate stiffness can induce undesirable heterogeneous cell morphologies, and therefore function, for a single cell type. For example, the morphology of epithelial breast cells is regulated by the stiffness of the culture surface. A concave meniscus effect can result in in vivo-like acini structures near the edge and monolayers near the center.

The curved coating surface created by the meniscus can also result in uneven cell distribution on the surface when seeding the cells. In some occasions, the cells tend to either cluster in the center of the wells or towards the edge of the wells due to gravity or material preference of the cells. This can be troublesome especially in applications where uniform single cell seeding is preferred.

In order to have a desired amount of coating in the center area of the bottom surface within a well when the surface is a concave meniscus, an excessive amount of coating or coating precursors is often used when applying the coating to the well to overcome the effect. While such excessive coating may result in less difference in mechanical properties (e.g., stiffness) between the center and edge, the upper surface of the coating remains curved. Therefore uneven cell distribution on the surface persists. Additionally the thick coating with curved upper surface creates multiple focal planes in one field of view (FOV). As a result, only a part of the field of view (FOV) can be in focus while the rest of the FOV is out of focus using conventional microscopy techniques. To view cells on such curved surfaces, confocal imaging techniques are often used to obtain a focused image of a full field of view at the expense of time and cost relative to conventional microscopy. In addition for coating precursors that require UV curing, excessive coating thickness in some areas can result in those areas not being fully cured. Any uncured precursors may later release into the culture medium and become toxic to the cells. Further, hydrophobic coating materials such as polydimethylsiloxane (PDMS) are known to extract small hydrophobic compounds from the culture medium. Therefore, excessive coating thickness can result in large quantities of small hydrophobic compounds being extracted, which can interfere with downstream cell based assays such as toxicity evaluations of pharmaceutical compounds. Additionally, excessive coating thickness may be cost prohibitive, particularly when expensive coating materials are employed.

The cell culture articles and methods disclosed herein aim, among other things, to provide a solution to achieve an even coating on the bottom surface of a container for tissue culture, especially when only a small quantity of coating or coating precursors is desired.

### BRIEF SUMMARY

The present disclosure describes, among other things, cell culture devices having a surface with micro-pillar structures and a thin layer of coating material, which forms a continuous surface of uniform chemistry and mechanical properties that enables homogeneous in vivo-like cell culture as well as uniform cell distribution.

In various embodiments, an article for culturing cells includes a substrate having a surface; a plurality of pillars extending from the surface of the substrate; and a polymeric coating disposed on the surface of the substrate between the pillars, forming a plurality of mini-menisci on the surface of the substrate between the plurality of pillars. The mini-menisci have diameters of from about 20 micrometers to about 250 micrometers. The plurality of pillars are spaced apart from one another in a manner to encourage cell growth on the mini-menisci rather than the on top of the pillars. Methods of culturing cells on such articles and methods of manufacturing such articles are also described herein.

One or more embodiments of the cell culture articles or methods described herein provide one or more advantages over prior cell culture articles or methods related to cell culture or cell culture articles. For example, embodiments of cell culture articles described herein address the meniscus effect on a macroscale by forming a plurality of local mini-menisci, which results in a more uniform surface on the macroscale. A small quantity of coating materials may be sufficient to form a uniform coating on a surface having micro-pillar structures as described in embodiments herein. The pillars localize the meniscus effect between the pillars on the surface and transform a single meniscus on a flat surface into many continuous mini-menisci on a structured bottom surface. These mini coating menisci may be uniform in both chemistry and mechanical properties, serving as a way to either create a very uniform coating on the bottom of the well or by using regular geometrical structures of pillars on an intermediate scale to create mini-menisci that form mini-wells or pockets to house cells for culture. As a result, the cells cultured on these surfaces may develop homogeneous morphologies and function. By way of further example, the capability of creating a uniform coating on the bottom surface of a well using a small quantity of coating material reduces manufacturing cost, particularly when expensive coating materials are used. In addition, the capability of creating a uniform coating on a surface of a cell culture article using a small quantity of coating material makes it feasible to use materials that may take up hydrophobic materials from the culture medium into the coating. Therefore, materials such as PDMS, may now be more practical for cell culture applications that involve the use of hydrophobic compounds because of reduced extraction of the hydrophobic compound by the PDMS due to the reduced amount of PDMS used. Additionally, single cell seeding can be achieved by controlling cell seeding density, particularly in embodiments, where intermediate scale mini-menisci are formed between pillars. Furthermore, the mini-menisci formed between pillars may enable development of many uniform cell aggregates or spheroids to form from single cells within one mini-meniscus rather than one large clump as often formed on a coated flat bottom well. Also, interconnected mini-menisci having uniform dimensions may be created by the pillars in embodiments. This can enable the formation of cell aggregates, spheroids and tumors of a uniform size within the mini-menisci. These and other advantages will be readily understood from the following detailed description when read in conjunction with the accompanying drawings.

### BRIEF DESCRIPTION OF THE DRAWINGS

FIG. 1 is perspective view of a schematic cell culture article having a plurality of pillars extending from a surface of the article.

FIG. 2 is a perspective view of a sectioned schematic cell culture article having a plurality of pillars extending from a surface of the article.

FIG. 3 is a top down view of a schematic illustration of a portion of a cell culture article having pillars extending from a surface.

FIG. 4 is a side view of a schematic illustration of a portion of a cell culture article having pillars extending from a surface and a coating forming mini-menisci on the surface between the pillars.

FIGS. 5A-5C are top-down microscopic images of cell culture articles having pillars extending from a surface.

FIG. 6 is an X-ray micro Computed Tomography image of a cell culture article having pillars extending from a surface and a coating forming mini-menisci on the surface between the pillars.

FIG. 7 is an X-ray micro Computed Tomography image of a portion of a 96 well TCT plate having wells coated with various amounts a silicone hydrogel.

FIG. 8 is a microscopic image of cells cultured in a well of a traditional cell culture apparatus coated with PDMS.

FIGS. 9A-B are microscopic images of a cell culture article having a solid wall micro-wells before (9A) and after (9B) seeding with cells.

FIG. 10A is a schematic drawing of a repeating hexagonal structure (lattice) formed with pillars.

FIG. 10B is a schematic drawing of the hexagonal structures of FIG. 10A propagated into a circle of 6 mm in a 6×6 array generated to fit into 96 well holy plates.

FIG. 11 is a bar graph showing the amount of nefazodone remaining in culture medium after incubation with various amounts of PDMS.

FIGS. 12A-D are microscopic images of breast cancer cells cultured on various surfaces.

FIGS. 13A-C are microscopic images of primary human hepatocytes cultured on various surfaces.

FIG. 14 is a bar graph showing CYP3A activity of primary human hepatocytes cultured on the surfaces shown in FIGS. 13A-C.

FIGS. 15A-B are scanned images showing 2 μl, 5 μl, 10 μl, 20 μl, 50 μl of Matrigel™ coating on the bottom surface of a standard 96-well TCT plate (A) and a 96-well plate of PS substrate having micro-pillar structures (B).

FIGS. 16 A-C are fluorescence images showing endothelial tube formation on standard TCT surface coated with 0, 10, and 50 μl of Matrigel™ coating.

FIGS. 16D-F are fluorescence images showing endothelial tube formation on PS substrates having micro-pillar structures coated with 0, 10 and 50 μl of Matrigel™.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. As used herein, “consisting essentially of,” as it relates to a composition, coating, article, method, or the like, means that the components of the composition, coating, article, method, or the like are limited to the enumerated components and any other components that do not materially affect the basic and novel characteristic(s) of the composition, coating, article, method, or the like.

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

As used herein, “precursors of a polymeric material” are monomers, oligomers, polymers or the like that may be polymerized, cross-linked or the like to produce the polymeric material under appropriate reaction conditions, such as UV curing.

The present disclosure describes, among other things, cell culture devices having a surface with micro-pillar structures and a thin layer of coating material, which forms a continuous surface of uniform chemistry and mechanical properties that may enable homogeneous in vivo-like cell culture as well as uniform cell distribution.

Any suitable cell culture article may be modified to include micro-pillars and coatings as described herein. For example, single and multi-well plates, such as 6, 12, 96, 384, and 1536 well plates, jars, petri dishes, flasks, beakers, plates, roller bottles, slides, such as chambered and multichambered culture slides, tubes, cover slips, cups, spinner bottles, perfusion chambers, bioreactors, and fermenters may include a structure surface in accordance with the teachings provided herein. Such articles may be fabricated from any suitable base material, such as glass materials including soda-lime glass, pyrex glass, vycor glass, quartz glass; silicon; plastics or polymers, including dendritic polymers, such as poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), polysaccharide, polysaccharide peptide, poly(ethylene-co-acrylic acid) or derivatives of these; or the like; combinations thereof.

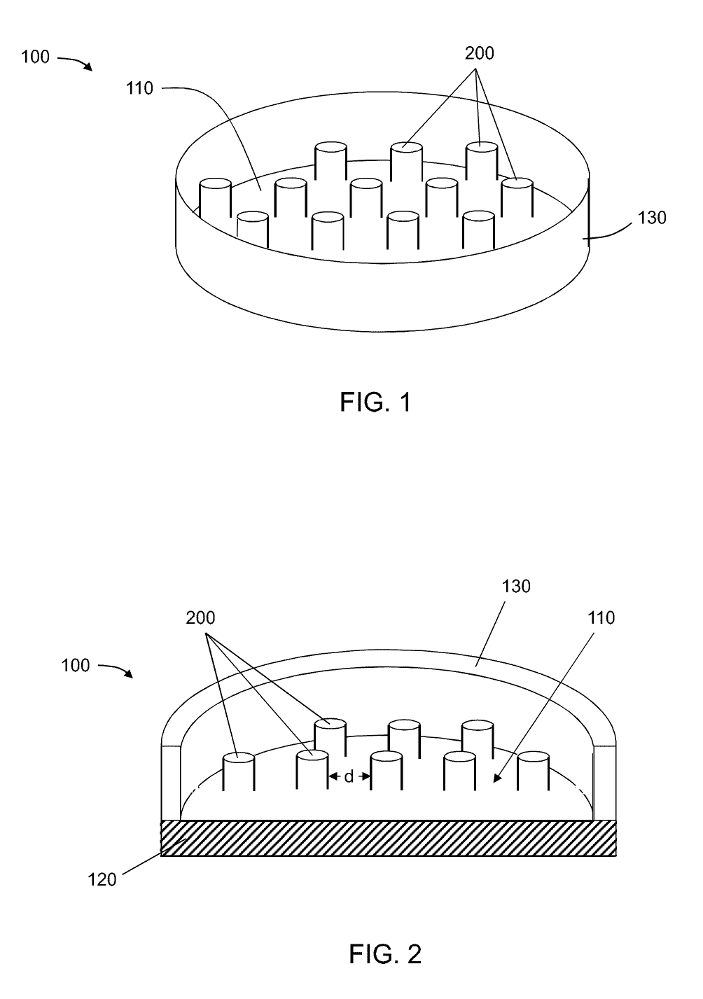

With reference to FIGS. 1-2, representative cell culture articles 100 to which a polymeric coating may be applied are shown. The cell culture articles 100 have a plurality of pillars 200 extending from a surface 110 of a base material or substrate 120 of the cell culture articles 100. In embodiments, a layer (not shown) is positioned between the base of the pillars 200 and the surface of the base material or substrate 120 of the article 100. The layer may be, for example, a part of a mold that forms the pillars. Of course, any suitable layer may be present. If a layer is present between the base of the pillars and the surface of the base material of the article, the “substrate” from whose surface the pillars extend is the layer. If more than one layer is present between the base of the pillars and the surface of the base material of the article, the “substrate” from whose surface the pillars extend is the top most layer.

In embodiments, the surface 110 and pillars 200 define a structured and highly reproducible three-dimensional geometry for culturing cells. Each pillar 200 may have defined geometric dimensions that are reproducible to a degree commensurate with the reproducibility of the processes employed to form the pillars 200.

The pillars 200 are configured or spaced apart to encourage cultured cells to grow on the coating (not shown in FIGS. 1-2) rather than on top of the pillars 200. For example, the pillars 200 may have a diameter sufficiently small to discourage cell attachment; e.g., the pillars 200 may have a diameter of about 15 micrometers or less or about 10 micrometers or less. In addition, or alternatively, the tops of the pillars 200 may be shaped to discourage cell attachment. For example, the tops of the pillars may be rounded or tapered. Each pillar 200 may have the same or different diameter as another pillar.

The pillars 200 may be spaced apart sufficiently such that pillars 200 cannot readily support cells. By way of example, cells are typically about 10 micrometers in diameter. If the pillars 200 are spaced apart such that no two are closer than 10 micrometers apart (from the periphery of one pillar to the periphery of another pillar), cells cannot be readily supported on top of the pillars; e.g., the pillars may be 15 micrometers or more apart or 20 micrometers or more apart. In some embodiments, some pillars 200 are closer than 10 micrometers apart (or 15 or 20 micrometers apart), but in any group of three pillars the distance between at least two of the pillars is more than 10 micrometers apart, such as 15 or 20 micrometers apart.

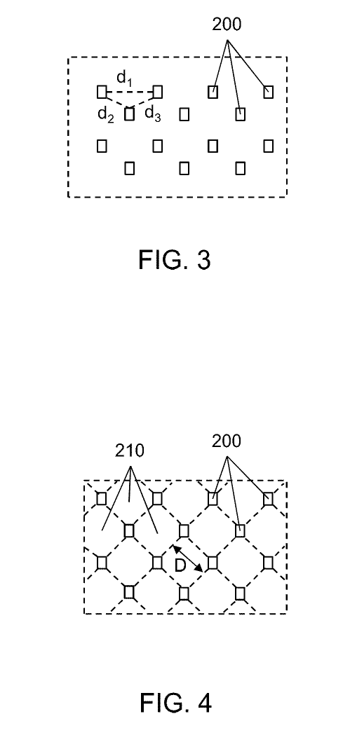

For example and with reference to FIG. 3, at least one of the distances (d₁, d₂, d₃) between the group of three pillars 200 is greater than about 10 micrometers (e.g., about 15 micrometers or greater or about 20 micrometers or greater). For example, d₂ and d₃ may be less than 10 micrometers (or 15 micrometers or 20 micrometers) as long as d₁ is more than 10 micrometers (or 15 micrometers or 20 micrometers). With such arrangement and spacing of pillars, cell growth or attachment on top of pillars may be avoided or minimized, with cell growth occurring on the coating surface between the pillars.

For example and with reference to FIG. 4, because of the size and shape or spacing of pillars 200, cells are encouraged to grow on a coating disposed on a surface between the pillar. The coating forms mini-meniscus structures 210 between the pillars 200. The mini-mensci structures 210 may be concave or convex. The overall shape of the concave or convex mini-menisci 210 will be determined in part by the number of pillars 200 between which the mini-menisci 200 are formed. In the depicted embodiment, the mini-menisci 210 are formed between four pillars 200. Of course, the mini-menisci structures 210 may be formed between any suitable number of pillars, such as three or more, four or more, five or more, etc., depending on the spacing of the pillars 200. In the embodiment depicted in FIG. 4, the pillars 200 and mini-menisci 210 are evenly distributed across the surface of the substrate. Of course, the pillars or mini-menisci may be unevenly distributed. In embodiments, one or more mini-menisci have different shapes or sizes. For example, mini-menisci of a given cell culture article may be formed between a different number of pillars or formed between pillars that are spaced differently.

The mini-menisc±210 may have any suitable shape and dimension. In embodiments, a mini-meniscus 210 a diameter D (see e.g., FIG. 4) of about 20 micrometers or more, such as from about 20 micrometers to about 250 micrometers, from about 20 micrometers to about 200 micrometers, or from about 80 micrometers to about 150 micrometers.

The mini-mensci are interconnected by polymeric material of the coating between the pillars. For example and still referring to FIG. 4, an edge of one mini-meniscus 210 forms an edge of an adjacent mini-meniscus 210. If the mini-menisci 210 are concave, a mini-meniscus has a low point generally towards the center of the mini-meniscus and higher points at the edges, where the height continually increases from the low point to higher points at the edges.

The pillars may be of any suitable height. The height of the pillars may affect the height of the edges of the mini-menisci formed between the pillars, which can affect the ability of the mini-menisci to isolate cells. For example, higher pillars tend to result in higher edges of menisci, which tend to increase the ability of mini-menisci to isolate cells. With lower pillars and lower meniscus edges, the cells may be more likely to move between mini-menisci during culture or seeding. In embodiments, the height of the pillars is from about 25 micrometers to about 200 micrometers, such as rom about 30 micrometers to about 120 micrometers, or from about 50 micrometers to about 110 micrometers. Each of the pillars may have the same or different height as another pillar.

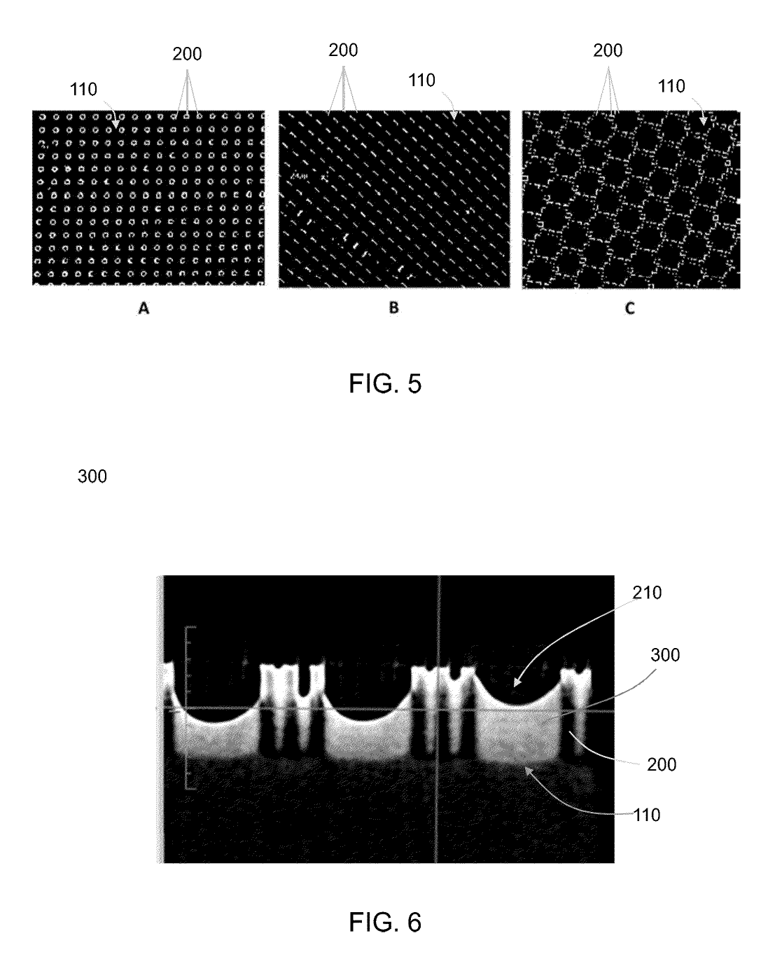

The pillars may be of any suitable cross-sectional shape. Each pillar may have the same or different cross-sectional shape as another pillar. For example and with reference to FIGS. 5A-C, three different pillar arrangements across surfaces of three different cell culture articles on which a coating may be disposed are shown. The pillars 200 extend from the surface 110 of the article, upon which a polymeric coating may be disposed. In FIG. 5A, the pillars have a round cross-sectional shape. In FIGS. 5B and 5C, the pillars have square cross-sectional shapes. The pillars in FIGS. 5A and 5B, the pillars are evenly distributed across the surface of the article. In FIG. 5C, the pillars form hexagonal arrays that are evenly distributed across the surface. In FIG. 5C, the pillars are arranged to form mini-meniscus structures that form micro-wells or micro-cups (if the meniscus is concave) of intermediate scale. Such micro-cup or micro-well menisci create a surface that enables single cell seeding and formation of cell spheroids.

The micro-well or micro-cup mini-menisci that may be formed on an article as depicted in FIG. 5C or on another suitable structured surface having pillars may be of dimensions similar to know micro-wells. However, micro-wells formed between pillars (e.g., pillars arranged as depicted in FIG. 5C) may have one or more advantage relative to micro-wells formed from solid walls. As described in more detail in the Examples, micro-wells formed from solid walls that are coated with hydrophilic polymers, such as PDMS, tend to trap air bubbles when seeded with cells in cell culture medium. To avoid the trapping air bubbles, the surface of such coated micro-wells must be wetted prior to seeding. In contrast, micro-wells of similar dimensions formed from mini-menisci between pillars as described herein do not suffer from the tendency to trap air bubbles and thus do not need to be pre-wetted prior to seeding or culture.

Referring now to FIG. 6, an X-ray microtomographic image of a cell culture apparatus having mini-menisci 210 is shown. The mini-menisci 210 are formed from polymeric coating 300 disposed on the surface 110 and between pillars 200.

The coating disposed on the surface of the article between the pillars may be any polymeric material suitable for culturing cells. By way of example, the coating may be formed from a hydrogel coating or a polymeric coating having high equilibrium water content. Examples of suitable polymeric coatings are described in US 2009/0191632, entitled SWELLABLE (METH)ACRYLATE SURFACES FOR CULTURING CELLS IN CHEMICALLY DEFINED MEDIA and US 2009/0191627, entitled SYNTHETIC SURFACES FOR CULTURING CELLS IN CHEMICALLY DEFINED MEDIA. In embodiments, the polymeric material forming the coating is hydrophobic. For example, the coating may comprise PDMS, polyurethane, epoxy, -polyester, or polypropylene.

The coating may be applied to or formed on the surface of the article in any suitable manner. In embodiments, the polymeric material of the coating is dissolved in a suitable solvent and applied to the surface of the cell culture article substrate. The solvent may be removed; e.g., by evaporation, leaving the coating forming the mini-menisci between the pillars. In embodiments, precursors of the polymeric material are disposed on the article and the polymeric material is formed on the surface of the substrate; e.g., by curing, resulting in the coating forming the mini-menisci between the pillars. Any suitable method for polymerizing or crosslinking precursors may be employed.

Any suitable amount of polymeric material may be applied to the article to form the mini-menisci coating. In embodiments, the mass of the polymeric coating per surface are of the surface of the substrate of the cell culture article is from about 0.0006 mg/mm² to about 1.6 mg/mm²; e.g, from about 0.003 mg/mm² to about 0.006 mg/mm² or from about 0.03 mg/mm² to about 0.06 mg/mm².

The thickness of the polymeric coating will vary depending on the amount of polymeric material applied. The amount of polymeric material applied will vary depending on the height and spacing of the pillars. In embodiments, the average thickness of the polymeric coating on the surface of the substrate is from about 0.6 micrometers to about 1500 micrometers; e.g, from about 6 micrometers to about 60 micrometers or from about 100 micrometers to about 1000 micrometers.

The pillars or an array of pillars may be formed via any suitable technique. For example, the pillars may be formed via a master, such as a silicon master. The master may be fabricated from silicon by proximity U.V. photolithography. By way of example, a thin layer of photoresist, an organic polymer sensitive to ultraviolet light, may be spun onto a silicon wafer using a spin coater. The photoresist thickness is determined by the speed and duration of the spin coating. After soft baking the wafer to remove some solvent, the photoresist may be exposed to ultraviolet light through a photomask. The mask's function is to allow light to pass in certain areas and to impede it in others, thereby transferring the pattern of the photomask onto the underlying resist. The soluble photoresist is then washed off using a developer, leaving behind a protective pattern of cross-linked resist on the silicon. At this point, the resist is typically kept on the wafer to be used as the topographic template for molding the stamp. Alternatively, the unprotected silicon regions can be etched, and the photoresist stripped, leaving behind a wafer with patterned silicon making for a more stable template. The lower limit of the features on the structured substrates is dictated by the resolution of the fabrication process used to create the template. This resolution is determined by the diffraction of light at the edge of the opaque areas of the mask and the thickness of the photoresist. Smaller features can be achieved with extremely short wavelength UV light (˜200 nm). For submicronic patterns (e.g. etch depths of about 100 nanometers), electron beam lithography on PMMA (polymethylmetacrylate) may be used. Templates can also be produced by micromachining, or they can be prefabricated by, e.g., diffraction gratings.

To enable simple demoulding of the master, an anti-adhesive treatment may be carried out using silanisation in liquid phase with OTS (octadecyltrichlorosilane) or fluorinated silane, for example. After developing, the wafers may be vapor primed with fluorinated silane to assist in the subsequent removal of the array of pillars. Examples of fluorinated silane that may be used include, but are not limited to, (tridecafluoro-1,1,2,2-tetrahydroctyl) trimethoxysilane, and tridecafluoro-1,1,2,2-tetrahydrooctyl) triethoxysilane.

In embodiments, the pillars are formed by injection molding.

Pillars may be made of any suitable polymeric material or inorganic material. Suitable inorganic materials include glass, silica, silicon, metal, or the like. Suitable polymeric materials include poly(dimethylsiloxane) (PDMS), a sol-gel, or other cell culture compatible polymer. Examples of suitable sol gels include sol gels formed through the hydrolysis of tetraethyl orthosilicate (TEOS) under acidic conditions. Other cell culture compatible polymers include polyesters of naturally occurring α-hydroxy acids, polyglycolic acid (PGA), poly(-lactic acid) (PLLA) and copolymers of poly(lactic-co-glycolic acid) (PLGA), amino-acid-based polymers, a polysaccharide, or polystryrene. The materials for forming pillars may be chosen based on desired mechanical, cell-interacting, or other properties for optimizing cell culture for distinct types of cells.

Pillars may be made of the same material as the substrate from which they extend or may be made of different material from the substrate. The pillars or substrate can be porous, non-porous, or macroporous. Pillars or substrates may be treated or coated to impart a desirable property or characteristic to the treated or coated surfaces. Examples of surface treatments often employed for purposes of cell culture include corona or plasma treatment.

A polymeric coating, as discussed above, may then be applied to an article having a substrate with a surface and the pillars extending from the surface. As discussed above, the coating forms mini-menisci structures on the surface between the pillars. The coating may include an extracellular matrix (ECM) material, or an ECM may be disposed on or conjugated to the coating. Any suitable ECM matrial may be used, such as naturally occurring ECM proteins or synthetic ECM materials. The type of EMC selected may vary depending on the desired result and the type of cell being cultures, such as a desired phenotype of the culture cells. Examples of naturally occurring ECM proteins include fibronectins, collagens, proteoglycans, and glycosaminoglycans. Examples of synthetic materials for fabricating synthetic ECMs include polyesters of naturally occurring α-hydroxy acids, poly(DL-lactic acid), polyglycolic acid (PGA), poly(-lactic acid) (PLLA) and copolymers of poly(lactic-co-glycolic acid) (PLGA). Such thermoplastic polymers can be readily formed into desired shapes by various techniques including moulding, extrusion and solvent casting. Amino-acid-based polymers may also be employed in the fabrication of an ECM for coating a pillar or substrate. For example, collagen-like, silk-like and elastin-like proteins may be included in an ECM. In various embodiments, an ECM includes alginate, which is a family of copolymers of mannuronate and guluronat that form gels in the presence of divalent ions such as Ca²⁺. Any suitable processing technique may be employed to fabricate ECMs from synthetic polymers. By way of example, a biodegradable polymer may be processed into a fiber, a porous sponge or a tubular structure. One or more ECM material may be incorporated in the polymeric coating or may be disposed on or conjugated to the coating that forms the mini-menisci.

Cell adhesion factors, such as polypeptides capable of binding integrin receptors including RGD-containing polypeptides or growth factors can be incorporated in the polymeric coating or may be disposed on or conjugated to the coating that forms the mini-menisci or may be incorporated into, form a part of, or be disposed on ECM, if present. Examples of adhesion polypeptides that may be employed are described in, for example, US 2009/0191632, entitled SWELLABLE (METH)ACRYLATE SURFACES FOR CULTURING CELLS IN CHEMICALLY DEFINED MEDIA and US 2009/0191627, entitled SYNTHETIC SURFACES FOR CULTURING CELLS IN CHEMICALLY DEFINED MEDIA.

Cell culture articles having mini-menisci as described above may be used to culture a variety of cells, such as breast cancer cells, hepatocytes, stem cells and the like. Cells may be seeded on the surface at any suitable density. Typically, cells are seeded at a density of between about 100 cells per square millimeter of surface area and about 5000 cells per square millimeter of surface area of the article. The seeding density can be optimized, based on culture conditions and duration. For example, for long term culture, the seeding density can be lower (e.g., 100 cells to 2000 cells per square millimeter of surface area of the article). Furthermore, if it is desired to seed one cell per mini-meniscus, it may be desirable to use lower seeding densities.

Any suitable incubation time and conditions may be employed in accordance with the methods described herein. It will be understood that temperature, CO₂ and O₂ levels, culture medium content, and the like, will depend on the nature of the cells being cultured and can be readily modified. The amount of time that the cells are incubated on the surface may vary depending on the cell response being studied or the cell response desired. Prior to seeding cells, the cells may be harvested and suspended in a suitable medium, such as a growth medium in which the cells are to be cultured once seeded onto the surface. For example, the cells may be suspended in and cultured in serum-containing medium, a conditioned medium, or a chemically-defined medium. The optimal culture medium for each type of cells, such as recommended by American Tissue Cell Culture or other suppliers, can be used with or without modifications.

A number of embodiments of cell culture articles and methods are described herein. A summary of selected aspects of such compositions and methods is provided below.

In a first aspect, an article for culturing cells includes a substrate having a surface, a plurality of pillars extending from the surface of the substrate; and a polymeric coating disposed on the surface of the substrate between the pillars, forming a plurality of mini-menisci on the surface of the substrate between the plurality of pillars. The mini-menisci have diameters of from about 20 micrometers to about 250 micrometers. The plurality of pillars are spaced apart from one another in a manner to encourage cell growth on the mini-menisci rather than the on top of the pillars.

A second aspect is an article of the first aspect, wherein the mini-menisci are evenly distributed across the substrate surface.

A third aspect is an article of the first aspect, wherein the pillars are distributed evenly across the substrate surface.

A fourth aspect is an article of any of the preceding aspects, wherein the mini-menisci are interconnected by the polymeric material between pillars

A fifth aspect is an article of any of the preceding aspects, wherein each of the mini-menisci are formed between four pillars.

A sixth aspect is an article according to any of aspects 1-4, wherein each of the mini-menisci are formed between more than four pillars.

A seventh aspect is an article according to any of the preceding aspects, wherein the mini-menisci have a diameter from about 20 micrometers to about 200 micrometers.

An eighth aspect is an article according to any of the preceding claims, wherein the mini-menisci have a diameter from about 80 micrometers to about 150 micrometers.

A ninth aspect is an article according to any of the preceding aspects, wherein the pillars have a diameter of about 15 micrometers or less.

A tenth aspect is an article according to any of the preceding aspects, wherein the pillars have a diameter of about 10 micrometers or less.

An eleventh aspect is an article according to any of the preceding aspects, wherein the polymeric coating comprises a hydrophobic polymer.

A twelfth aspect is an article according to any of the preceding aspects, wherein the polymeric coating comprises polydimethylsiloxane.

A thirteenth aspect is an article according to any of the preceding aspects, wherein the polymeric coating consists essentially of polydimethylsiloxane.

A fourteenth aspect is an article according to any of the preceding aspects, wherein the mass of polymeric coating per surface area of the surface of the substrate is from about 0.0006 mg/mm² to about 1.6 mg/mm²

A fifteenth aspect is a method comprising culturing a cell on a mini-meniscus of a cell culture article according to any of the preceding aspects.

A sixteenth aspect is a method according to the fifteenth aspect, wherein the cell is seeded on the article at a cell density sufficiently low to result in the cell being the only cell cultured on the mini-meniscus.

A seventeenth aspect is a method for making an article according to any of aspects 1-13, comprising: (i) providing an article having the substrate and the plurality of pillars; and (ii) disposing the polymeric coating on the surface of the substrate between the pillars to produce the plurality of mini-menisci on the surface of the substrate between the plurality of pillars.

An eighteenth aspect is a method according to the seventeenth aspect, wherein the article having the substrate and the plurality of pillars is produced by injection molding.

A nineteenth aspect is a method according to the seventeenth aspect, wherein the article having the substrate and the plurality of pillars is produced by attaching a structure comprising the plurality of pillars to the surface of the article.

A twentieth aspect is a method for making an article according to any of aspects 1-13, comprising: (i) providing an article having the substrate and the plurality of pillars; (ii) disposing precursors of the polymeric coating on the surface of the substrate between the pillars; and (iii) curing the polymeric precursors to produce the polymeric material having the plurality of mini-menisci on the surface of the substrate between the plurality of pillars.

In the following, non-limiting examples are presented, which describe various embodiments of the articles and methods discussed above.

### EXAMPLES

### Example 1

### The Meniscus Effect on Traditional Cell Culture Articles

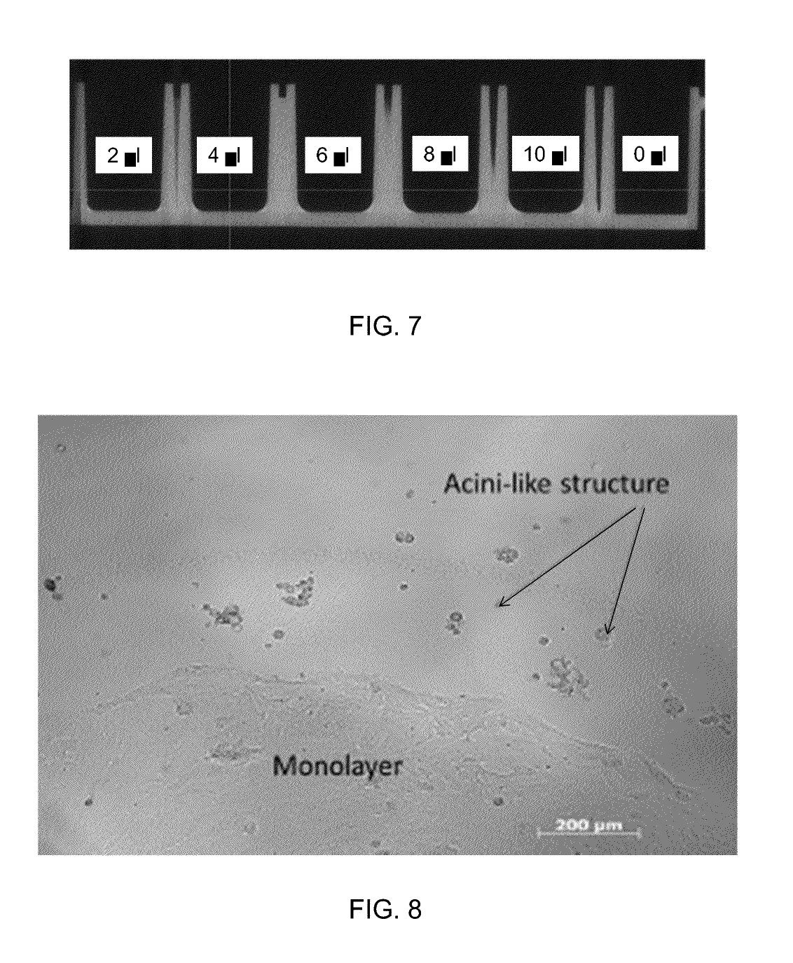

A 96-well TCT plate was coated with various amounts (0 μl, 2 μl, 4 μl, 6 μl, 8 μl, or 10 μl) of a silicone hydrogel to illustrate the meniscus effect with traditional cell culture articles. An X-ray microtomograph was taken of wells of the coated plate. The results are shown in FIG. 7 (25 μm spatial resolution). As can be seen, at all coating amounts, the meniscus effect is pronounced with very little coating present at the middle of the menisci. Accordingly, the effective stiffness at the middle of the meniscus is assumed to be more like the underlying hard plastic (polystyrene) and the stiffness at the edges is assumed to be more like the soft hydrogel. The dynamic substrate stiffness can induce undesirable heterogeneous cell morphologies, and therefore function, for a single cell type.

### Example 2

### Effects of the meniscus effect of traditional culture articles on cells

Because the morphology of epithelial breast cells is known to be regulated by the stiffness of the culture surface, breast cancer cells (MCF-10A) were cultured on TCT plate coated with 0.0063 mg/mm² of PDMS. As shown from the microscopic image in FIG. 8, two distinct cell morphologies were formed on the concave meniscus. In vivo-like acini structures formed towards the edge (on the softer portion) while a monolayer formed toward the center of the well (on the stiffer portion).

### Example 3

### Air Bubble Entrapment of PDMS Coated Microwells

In theory, coated micro-wells formed on a surface of a cell culture article and having solid walls (as opposed to pillars) to contain the coating can be used to localize meniscus effects to achieve coating uniformity (on a macro-scale) in a manner similar to the mini-menisci formed between pillars as described herein. However, when the coating material is hydrophobic, air bubbles are easily trapped in the micro-wells formed by solid walls due to surface tension when exposed to culture media during cell seeding, thus preventing cells from settling into the micro-wells.

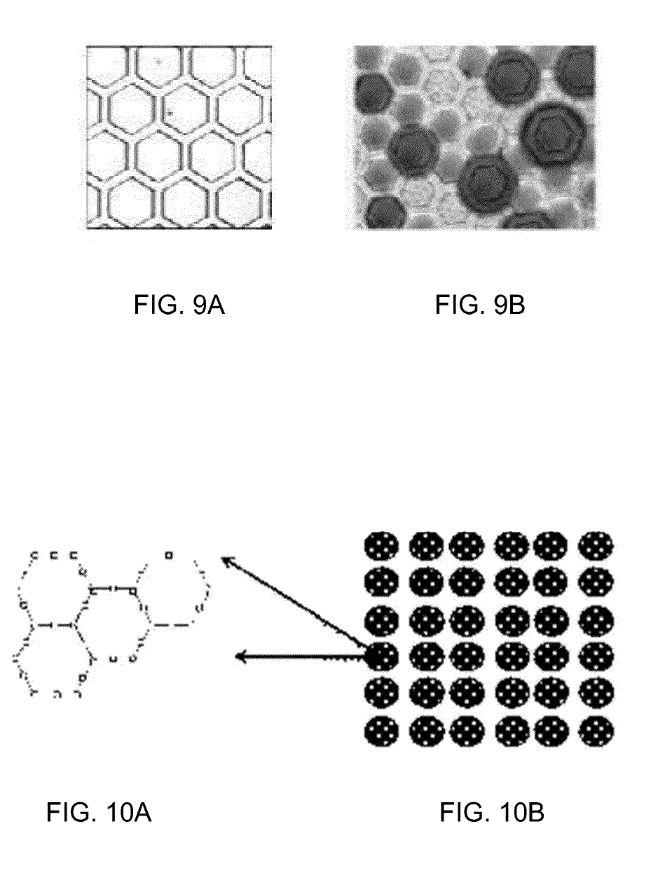

As shown in FIG. 9A (prior to seeding) and FIG. 9B (after seeding), micro-wells having solid walls and coated with PDMS tend to trap air bubbles when cells are seeded. Accordingly, a wetting procedure for such substrate is required in order to achieve even cell seeding on the culture surface. In contrast, similar coated structures formed between pillars as described herein did not trap air bubbles (see FIG. 12D, which is described in more detail below). A cell culture article having micro-wells as depicted in FIGS. 9A-B was formed as follows. Briefly, a 2% PDMS coating solution was prepared by dissolving in hexane solvent a mixture of PDMS pre-polymer A and B (commercially available Sylgard184 kit) in a ratio of 9 to 1. 10 μl of such coating solution was dispensed in each well of a 96-well plate and allowed hexane to evaporate in a few minutes. The coating was cured at room temperature or at 60° C. for 1 hour.

When compared to micro-wells formed between micro-pillars, the solid edge of the micro-well structures (e.g, as in FIGS. 9A-B) amounts for a higher surface contact area with coating materials. Therefore more material is contained in the meniscus and a higher meniscus height occurs at the edges. Therefore, there is less coating in central area of the micro-wells than is found in wells created by micro-pillars given the same amount of coating material. To create a similar central coating thickness, a larger amount of coating material is needed for a substrate surface having solid wall micro-well structures than those made of micro-pillars as described herein.

### Example 4

### Fabrication of Culture Article

Micro-pillar patterns were created on silicon wafers using standard photolithography and reactive ion etching techniques. For example, a hexagonal structure (lattice) can be formed with pillars (FIG. 10A) of 20-micron squares in size and 80-150-micron in height. The repeating lattice distance (or diameter of resulting “microwell”) ranged from 80-140 micron. The hexagonal structures propagated into a circle of 6 mm and a 6×6 array of these circles were generated to fit into 96 well holy plates (FIG. 10B). Alternatively, a “carpet” of micropillars can be formed of uniform height and spacing depending on the desired coating thickness (see, e.g., FIGS. 5A-B).

Briefly, the patterns were designed in CAD and generated in a chrome mask using Heidelberg Mask Writer DWL2000. Photoresist Shipley 1827 was spin-coated onto silicon wafers and exposed through the mask in an ABM's High Resolution Mask Aligner. After development in alkaline solution, the exposed photoresist was washed off leaving patches of patterns on silicon wafers. The wafers were then cleaned in an Oxford 81 Etcher and dry-etched in a PlasmaTherm SLR-770 ICP Etcher using the Bosch™ process. The remaining photoresist was stripped off in a Gasonics Aura 1000 downstream washer. The negative replica of such pillar patterns was transferred from the silicon masters to elastic molds made of PDMS (prepared using a Dow Sylgard 184 kit) using the cast-and-cure method, and then the pillar patterns were transferred from the PDMS mold to Polystyrene (PS) substrate through a hot embossing process at 200° C. and 2700 Psi. The PS substrates with the micro pillar patterns were sealed onto the back of 96-well holy plates using double adhesive tape.

A thin layer of PDMS coating was applied to the pillar structures by dispending 2 μl of 1% PDMS pre-polymer solution in hexane (equivalent to 0.02 mg of PDMS) into each well. Hexane solvent was allowed to evaporate (in the hood), and finally, the PDMS pre-polymers were cured at room temperature or at 60° C. for 1 hour.

### Example 5

### Compound Extraction (Loss) from Culture Medium is Negligible

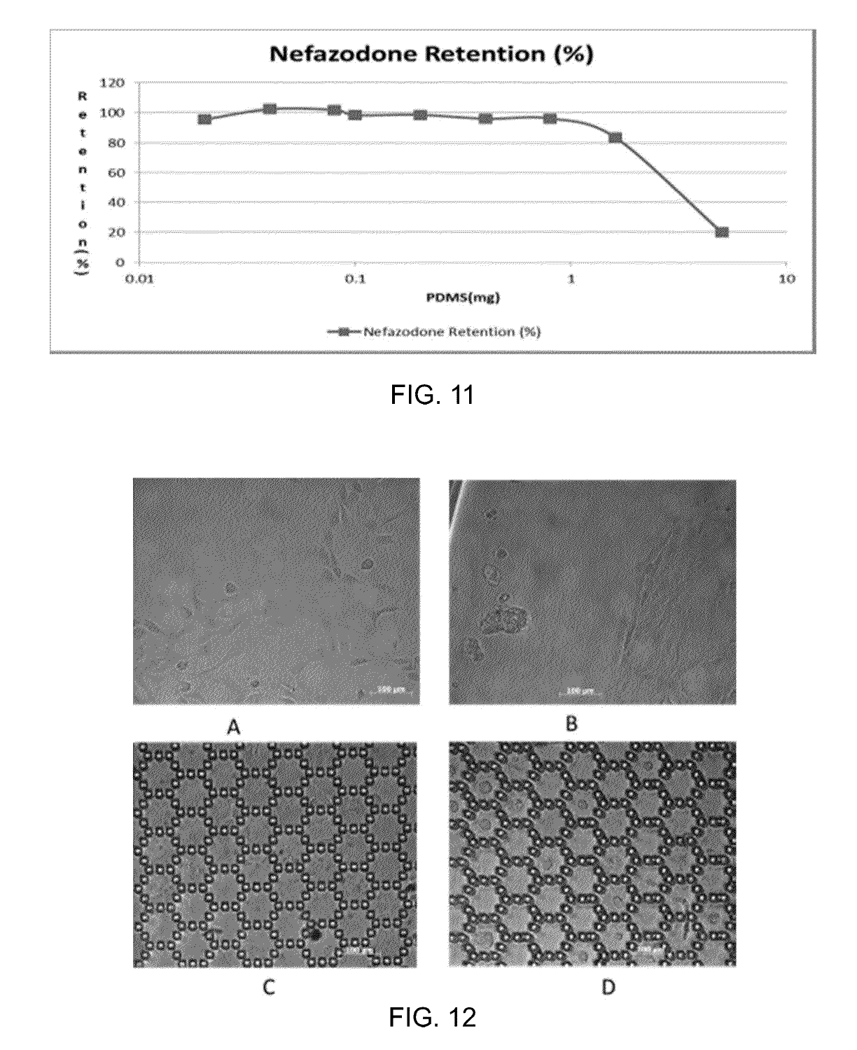

Nefazodone is a small hydrophobic compound that can be readily extracted from aqueous buffer by PDMS following a partition function rule, which means the loss of Nefazodone decreases with the amount of PDMS present in a given volume of buffer. In order to determine the safe quantity of PDMS for use with 200 μL of PBS buffer, 200 μL of a 200 μM Nefazodone-PBS solution was incubated in wells (formed generally as described above in EXAMPLE 4) containing varying PDMS coating amounts from 0.005 mg to 4 mg per well for 24 hours. After 24 hours the Nefazodone that remained in the PBS buffer was measured by UV-VIS adsorption at 260 nm. The results shown in FIG. 11 indicate that the loss of Nefazodone is negligible in the presence of less than 0.8 mg of PDMS in 200 μL of PBS buffer (meaning volume ratio of PDMS to buffer is less than 1/250). Therefore, a device having only 0.02 mg of PDMS coating in each well of a 96-well plate does not interfere with cell applications that require the use of hydrophobic compounds including Nefazodone. Such devices, formed as described herein as mini-menisci on a surface between micro-pillars, are suitable for culturing a variety of cells such that the cells exhibit in-vivo-like morphology.

### Example 6

### Formation of In Vivo-Like Acini Structures of Breast Cells

MCF-10A breast cells were cultured in a standard culture medium on standard TCT surface(flat bottom), TCT (flat bottom) coated with 0.02 mg of PDMS, PS substrates having micro-pillar structure with no coating, and PS substrate having micro-pillar structures coated with 0.02 mg PDMS. The micro-pillar structures were as described above with regard to EXAMPLE 4 Standard Culture Medium Contains Basal Medium DMEM/F12 (invitrogen#11965-118) with 5% horse serum (invitrogen #16050-122), 5 ml of Pen/Strep (100× solution, Invitrogen #15070-063), 20 ng/ml of EGF, and 0.5 ug/ml of Hydrocortison, 100 ng/ml of Cholera toxin and 10 ug/ml of insulin.

The cell morphologies that developed in the four substrates were distinctively different as shown in FIGS. 12A-D. FIG. 12A shows the cells spread and formed a monolayer morphology on the standard TCT flat bottom surface; on the flat bottom surface coated with 0.02 mg of PDMS, the cell morphology as shown in FIG. 12B was mixed: acini-like structures were formed near the edge while a monolayer formed towards the center of the well. Cells spread on the PS substrate surface having micro-pillar structures but no PDMS as shown in FIG. 12C, while on the surface with both micro-pillar structures and 0.02 mg of PDMS coating, the MCF-10A cells formed acini structures developed from single cells in each pocket as shown in FIG. 12D.

### Example 7

### Enhanced CYP3a Functions of Primary Human Hepatocytes

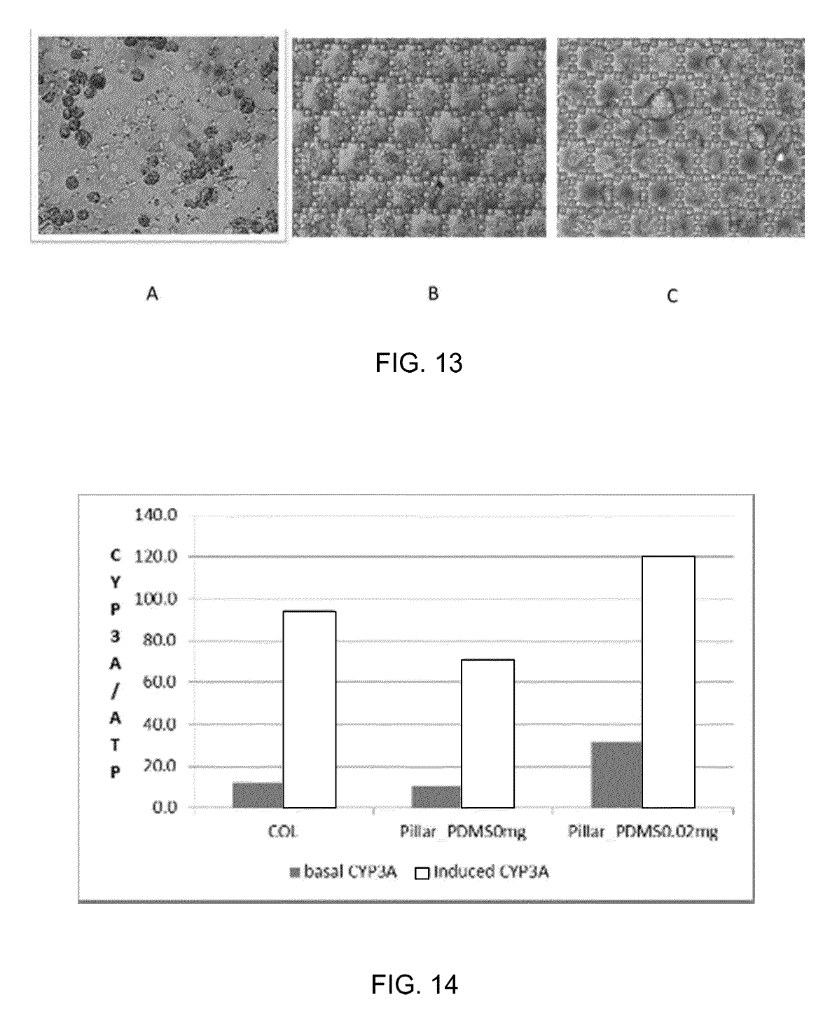

Primary human hepatocytes were seeded and cultured in standard 96-well TCT (flat bottom) plates with a collagen coating, a PS substrate having micro-pillar structures with no coating and PS substrates having micro-pillar structures with a 0.02 mg PDMS coating (as described above in EXAMPLE 4). As shown in FIG. 13A, the primary hepatocytes formed monolayers on the collagen surface. On the surface with micro-pillar structures but no coating, the hepatocytes formed loose aggregates in each pocket (FIG. 13B), while the PDMS coating on the pillars further enables cell/cell aggregation forming spheroid structures (FIG. 13C). This alteration of cell morphology was also reflected in the basal CYP3A and induced CYP3A function of primary hepatocytes as shown in FIG. 14. The culture substrate having micro-pillar structures and a PDMS coating demonstrated greatly enhanced CYP3A functions for human primary hepatocytes.

### Example 8

### Less Coating Material Needed to Form Uniform Coating in Culture Wells

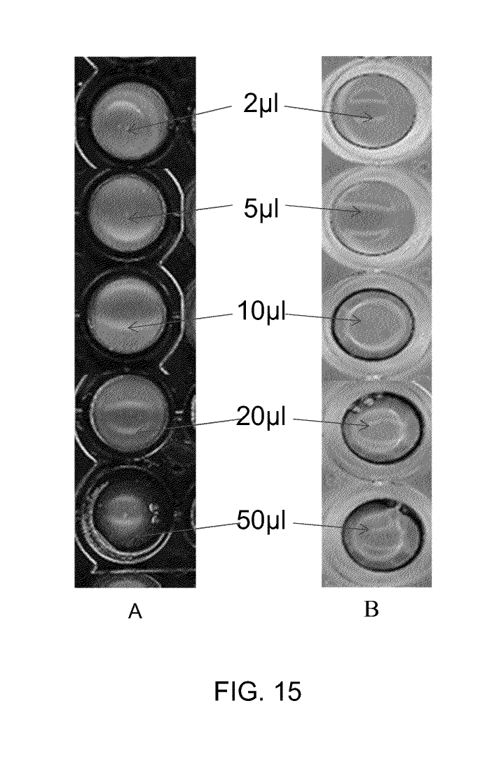

2 μl, 5 μl, 10 μl, 20 μl, and 50 μl of Matrigel™ (BD Biosciences) thawed overnight at 4° C. were dispensed in wells in a standard 96-well TCT plate (flat bottom) as well as wells in a 96-well plate of PS substrate having micro-pillar structures. As shown in FIG. 15, the same amount of Matrigel™ coating was able to spread more efficiently on substrate having micro-pillar structure than on flat substrate. As a result, no more than 10 μl of Matrigel™ is sufficient to create uniform coating on substrate having micro-pillar structure while 50 μl is needed on flat substrate. This represents a significant cost reduction when expensive coating materials such as Matrigel™ are used.

### Example 9

### Meniscus-Free Endothelial Tube Formation

One of the most widely used in vitro assays to model the reorganization stage of angiogenesis is the tube formation assay. The assay measures the ability of endothelial cells to form capillary-like structures (a.k.a tubes). Scientists typically employ this assay to determine the ability of various compounds to promote or inhibit tube formation. Tube formation is typically quantified by measuring the number, length, or area of these capillary-like structures in two-dimensional microscope images of the culture dish.

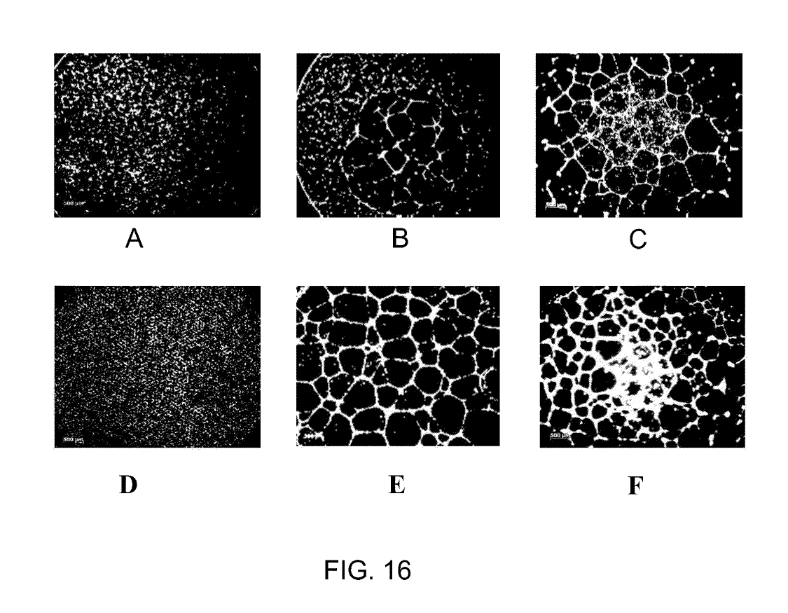

Primary human umbilical vein endothelial Cells (HUVE) were seeded and cultured in a standard 96-well TCT plate (flat bottom) having Matrigel™ coating of 0, 10 μl and 50 μl of and a 96-well plate of PS substrate having micro-pillar structures and Matrigel coating of 0, 10 μl and 50 μl. After 12 hours, the culture plates were stained using Calcein AM dye (BD Biosciences) and imaged using a fluorescence microscope (FIG. 16 A-F). Without Matrigel™ coating, only monolayer (no tube formation) was found in standard TCT plate or PS substrate having micro-pillar structures as shown in FIG. 16A and FIG. 16D. With 10 μl al of Matrigel™ coating in each well, tube formation was found only in the well center of in the standard TCT plate (FIG. 16B) while tube formation was found in the entire well on the PS substrate having micro-pillar (FIG. 16E); in addition, the tube formation in the entire well is in focus within one image. When 50 μl of Matrigel™ (recommended by vendor) was used, tube formation was observed in the entire well in the standard TCT plate (FIG. 16C) as well as PS substrate having micro-pillar structures (FIG. 16F). However, as shown in FIG. 16C, only tube formation in part of the well is in focus within one image due to the coating meniscus (in contrast to FIG. 16G in which cells were cultured on a substrate having micropillars). Therefore, in order to quantify tube formation in each well of a cell culture article that does not have micro-pillars and has a global scale meniscus effect, confocal fluorescence microscope imaging techniques in which a group of images are captured and then combined needs to be performed at greatly increased time and expense.

Thus, embodiments of CELL CULTURE SUBSTRATE HAVING UNIFORM SURFACE COATING are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. An article for culturing cells, comprising:
a substrate having a surface;
a plurality of pillars extending from the surface of the substrate; and
a polymeric coating disposed on the surface of the substrate between the pillars, forming a plurality of mini-menisci on the surface of the substrate between the plurality of pillars,
wherein the mini-menisci have diameters of from about 20 micrometers to about 250 micrometers, and
wherein the plurality of pillars are spaced apart from one another in a manner to encourage cell growth on the mini-menisci rather than the on top of the pillars.

2. An article according to claim 1, wherein the mini-menisci are evenly distributed across the substrate surface.

3. An article according to claim 1, wherein the pillars are distributed evenly across the substrate surface.

4. An article according to claim 1, wherein the mini-menisci are interconnected by the polymeric material between pillars.

5. An article according to claim 2, wherein the mini-menisci are interconnected by the polymeric material between pillars.

6. An article according to claim 3, wherein the mini-menisci are interconnected by the polymeric material between pillars.

7. An article according to claim 1, wherein each of the mini-menisci are formed between four pillars.

8. An article according to claim 3, wherein each of the mini-menisci are formed between four pillars.

9. An article according to claim 1, wherein each of the mini-menisci are formed between more than four pillars.

10. An article according to claim 3, wherein each of the mini-menisci are formed between more than four pillars.

11. An article according to claim 1, wherein the mini-menisci have a diameter from about 20 micrometers to about 200 micrometers.

12. An article according to claim 3, wherein the mini-menisci have a diameter from about 20 micrometers to about 200 micrometers.

13. An article according to claim 8, wherein the mini-menisci have a diameter from about 80 micrometers to about 150 micrometers.

14. An article according to claim 1, wherein the pillars have a diameter of about 15 micrometers or less.

15. An article according to claim 14, wherein the pillars have a diameter of about 10 micrometers or less.

16. An article according to claim 1, wherein the polymeric coating comprises a hydrophobic polymer.

17. An article according to claim 8, wherein the polymeric coating comprises a hydrophobic polymer.

18. An article according to claim 17, wherein the polymeric coating comprises polydimethylsiloxane.

19. An article according to claim 1, wherein the mass of polymeric coating per surface area of the surface of the substrate is from about 0.0006 mg/mm² to about 1.6 mg/mm².

20. A method for making an article according to any claim 1, comprising:
providing an article having the substrate and the plurality of pillars; and
disposing the polymeric coating on the surface of the substrate between the pillars to produce the plurality of mini-menisci on the surface of the substrate between the plurality of pillars.
